(19) United States
(12) Patent Application Publication
(10) Pub. No.: US 2019/0030170 A1
(43) Pub. Date: Jan. 31, 2019

(54) CANNABINOID FORMULATIONS WITH IMPROVED SOLUBILITY

(71) Applicant: Vireo Health LLC, Minneapolis, MN (US)

(72) Inventors: Kyle Kingsley, Minneapolis, MN (US); Siyeon Lee, Albany, NY (US); Eric Greenbaum, New York, NY (US)

(73) Assignee: Vireo Health LLC, Minneapolis, MN (US)

(21) Appl. No.: 15/592,135

(22) Filed: May 10, 2017

Related U.S. Application Data
(60) Provisional application No. 62/333,927, filed on May 10, 2016.

(51) Int. Cl. A61K 47/40; A61K 9/08; A61K 9/00; A61K 31/047; A61K 31/352

(52) U.S. Cl. CPC A61K 47/40; A61K 9/08; A61K 31/352; A61K 31/047; A61K 9/0019

(57) ABSTRACT

The present invention provides aqueous formulations containing at least one cannabinoid, such as, for example, tetrahydrocannabinol (THC), cannabidiol (CBD), and a sulfoalkyl ether cyclodextrin. The liquid formulations are substantially clear, sterilizable, and chemically and physically stable. The liquid formulations do not precipitate upon dilution with distilled water or other pharmaceutically acceptable liquid carrier. The sulfoalkyl ether cyclodextrin-containing formulation provides significant advantages over other cyclodextrin-containing formulations of cannabiniods. The formulation can be prepared in acidic, neutral and slightly basic medium while providing acceptable concentrations of at least one cannabinoid suitable for administration. An SAE-CD-containing formulation of at least one cannabinoid can be provided in liquid form or as a reconstitutable solid. Solutions can be made either dilatable or non-dilutable with water at room temperature or under conditions typically encountered in the clinic.

### STATEMENT REGARDING FEDERALLY SPONSORED RESEARCH

N/A

### FIELD OF THE INVENTION

The present invention relates generally to cannabinoid formulations and compositions with improved solubility profiles.

### BACKGROUND OF THE INVENTION

The cannabinoids are a class of molecules isolated from the cannabis sativa/indica plant. Over the last several decades the medical utility of the cannabinoids has received substantial attention. The cannabinoids, while medically useful, are extremely hydrophobic molecules and are difficult to work with and formulate. Therefore, there is a need in the field for a composition of cannabinoids that have increased solubility and are easier to formulate.

Cyclodextrins and their derivatives are widely used in liquid formulations to enhance the aqueous solubility of hydrophobic compounds. Cyclodextrins are cyclic carbohydrates derived from starch. The unmodified cyclodextrins differ by the number of glucopyranose units joined together in the cylindrical structure. The parent cyclodextrins contain 6, 7, or 8 glucopyranose units and are referred to as α-, β-, and γ-cyclodextrin respectively. Each cyclodextrin subunit has secondary hydroxyl groups at the 2 and 3-positions and a primary hydroxyl group at the 6-position. The cyclodextrins may be pictured as hollow truncated cones with hydrophilic exterior surfaces and hydrophobic interior cavities. In aqueous solutions, these hydrophobic cavities provide a haven for hydrophobic organic compounds, which can fit all, or part of their structure into these cavities. This process, known as inclusion complexation, may result in increased apparent aqueous solubility and stability for the complexed drug. The complex is stabilized by hydrophobic interactions and does not involve the formation of any covalent bonds

Chemical modification of the parent cyclodextrins (usually at the hydroxyl moieties) has resulted in derivatives with sometimes improved safety while retaining or improving the complexation ability of the cyclodextrin. Of the numerous derivatized cyclodextrins prepared to date, only two appear to be commercially viable; the 2-hydroxypropyl derivatives (HP-β-CD or HPCD), neutral molecules being commercially developed by Jannsen and others, and the sulfoalkyl ether derivatives (SAE-β-CD or SAE-CD), being developed by CyDex, Inc.

The SAE-CDs are a class of negatively charged cyclodextrins, which vary in the nature of the alkyl spacer, the salt form, the degree of substitution and the starting parent cyclodextrin. The sodium salt of the sulfobutyl ether derivative of beta-cyclodextrin, with an average of about 7 substituents per cyclodextrin molecule (SBE7-β-CD), is being commercialized by CyDex, Inc. (Kansas) as CAPTISOL® cyclodextrin. The anionic sulfobutyl ether substituent dramatically improves the aqueous solubility of the parent cyclodextrin. Reversible, non-covalent, complexation of drugs with the CAPTISOL® cyclodextrin generally allows for increased solubility and stability of drugs in aqueous solutions.

It has been reported that the relative increase in the solubility of a poorly soluble drug in the presence of an SAE-CD is a product of the binding constant and the molar concentration of SAE-CD present (Stella et al. in U.S. Pat. Nos. 6,046,177 and 5,874,418). Compounds usually exhibit a conventional type AL (‘A’ Linear) binding curve (Higuchi, T. and Connors, K. A. in “Advances in Analytical Chemistry and Instrumentation Vol. 4” (Reilly, Charles N. Ed., John Wiley & Sons., 1965, pp. 117-212)) when binding to an SAE-CD. In a typical type AL profile, the total solubility of the drug (y-axis) in water increases linearly with increasing concentrations of cyclodextrin present (x-axis). The data usually fits a straight line and rarely deviates from this relationship unless the particular compound (drug) being solubilized possesses an unexpected binding relationship with the SAE-CD. The y-intercept of a best-fit line through the data is equal to the theoretical intrinsic solubility of the drug in water.

Equations 1 and 2 generally describe the dynamic and reversible binding equilibrium, where the amount of drug, for example, in the complexed form is a function of the concentrations of the drug and cyclodextrin, and the equilibrium or binding constant, K1:1.

Drug+Cyclodextrim←K1:1→Complex Equation 1:

K 1:1=[Complex] [Drug] [Cyclodextrim] Equation 2

### SUMMARY OF THE INVENTION

As specified in the Background Section above, there is a need in the art for cannabinoid formulations and compositions with improved solubility profiles.

The invention provides a composition of matter comprising a sulfoalkyl ether cyclodextrin and at least one cannabinoid. In some emobodiments the sulfoalkyl ether cyclodextrin is Captisol. In some embodiments the cannabinoid is CBD. In some embodiments the cannabinoid is THC. In some embodiments the cannabinoid is a combination of THC and CBD and/or other cannabinoids.

The invention provides a method for improving the aqueous solubility of at elast one cannabinoid or combination of cannabinoids including the steps of forming an admixture of at least one cannabinoid and an SAE-CD in an aqueous solvent.

The invention provides a method for improving the stability of at least one cannabinoid including the steps of forming an admixture of at least one cannabinoid and an SAE-CD in an aqueous solvent.

The invention provides a method for treating a patient in need thereof with a pharmaceutically acceptable formulation comprising an admixture of at least one cannabinoid and an SAE-CD. In particular, the formulation may be a formulation for enteral or parenteral formulation. In a particular embodiment, the formulation is an intravenous, intramuscular, subcutaneous, intradermal, or intraperitoneal injectable formulation.

The invention provides a sulfoalkyl ether cyclodextrin (SAE-CD)-based formulation of one or more cannabinoids. In one embodiment, the cannabinoid is CBD. In one embodiment the cannabinoid is THC. In one embodiment the cannabinoid is a combination of THC and CBD. The invention provides a commercially viable formulation that can be prepared and stored in aqueous liquids at a wide range of physiologically acceptable pH values and concentrations of cannabinoid without significant precipitation of the CBD in vitro. The formulation is pharmaceutically stable with a wide range of buffers, saline, or the like.

The SAE-CD and CBD-containing formulation has a sufficiently high cannabinoid concentration and stability for use as a commercial product. The formulation can be prepared as a clear aqueous solution that is sterilizable by sterile filtration (for example, filter pore size of less than or equal to 0.22 μm) and other conventional methods. The liquid formulation is stable under a variety of storage conditions. The formulation can be administered by injection at a physiologically acceptable pH range. Depending upon the pH of the medium, the SAE-CD can be present in less than stoichiometric, stoichiometric, or greater than stoichiometric amounts with respect to the amount of the cannabinoid present and still provide a clear solution.

One aspect of the invention provides a clear liquid formulation comprising at least a therapeutically effective amount of a cannabinoid, such as CBD, THC, or other cannabinoid, or combinations thereof, and a sulfoalkyl ether cyclodextrin present in an amount sufficient to provide a clear solution and in some embodiments, avoid precipitation when diluted with a pharmaceutically acceptable liquid excipient composition. The formulation can be provided as a stock solution, which is diluted with a liquid carrier composition such as saline, plasma, or lactated Ringer's solution prior to administration to a subject. Alternatively, the formulation can be provided at a concentration of cannabinoid(s) and SAE-CD that is suitable for administration without dilution. Upon dilution with a pharmaceutically acceptable aqueous liquid carrier, the present formulations will not precipitate or will form less precipitate than a corresponding formulation not containing the SAE-CD. In some embodiments, the formulation does not require a surfactant in order to render the formulation suitable for dilution.

Another aspect of the invention provides a clear ready-to-inject liquid formulation comprising SBE7-β-CD and a cannabinoid, wherein the SBE7-β-CD is present in an amount of at least about 0.1-40% by wt., and the cannabinoid is present in amount of 1-5 mg/mL.

Another aspect of the invention provides a dilutable concentrated liquid formulation comprising SBE7-β-CD and a cannabinoid present in amount of greater than about 0.5 mg/mL and the SBE7-β-CD to cannabinoid ratio is greater than or equal to about 10-50.

Still another aspect of the invention provides a solid pharmaceutical composition comprising at least one cannabinoid, an SAE-CD and optionally at least one other pharmaceutical excipient. When this composition is reconstituted with an aqueous liquid it forms a liquid formulation that can be administered by injection or infusion to a subject. Alternatively, the solid can be used to formulate additional form factors such as tablets, nebulizable powders and the like. In some embodiments the formulation may be provided directly as a powder.

The invention also provides a method of administering a cannabinoid agent to a subject in need thereof, comprising the step of administering a liquid formulation comprising a sulfoalkyl ether cyclodextrin and at least one cannabinoid. The formulation can be administered as an injection, such as, for example, intravenously, subcutaneously, intradermally, intraperitoneally, or intramuscularly.

Specific embodiments of the methods of the invention include those wherein: 1) the liquid formulation is administered by injection or infusion; 2) the method further comprises the earlier step of mixing the SAE-CD and at least one cannabinoid, and optionally one or more ingredients, in a solution to form the liquid formulation; 3) the method further comprises the step of diluting the liquid formulation in a pharmaceutically acceptable liquid carrier prior to administration; 4) the method comprises the step of forming the liquid formulation by mixing a liquid carrier with a reconstitutable solid comprising the SAE-CD and at least one cannabinoid; 5) the liquid formulation is formulated as described herein; 6) the liquid formulation provides equivalent or improved chemical stability characteristics as compared to the presently marketed cannabinoid formulations.

The invention also provides methods of preparing an SAE-CD and cannabinoid liquid formulation.

Another aspect of the invention provides a kit comprising a first pharmaceutical composition comprising an SAE-CD and a second pharmaceutical composition comprising a cannabinoid.

### BRIEF DESCRIPTION OF THE DRAWINGS

FIG. 1 shows chemical structure and formula of exemplary cylcodextrins of the present invention.

### DETAILED DESCRIPTION

These and other systems, methods, objects, features, and advantages of the present disclosure will be apparent to those skilled in the art from the following detailed description of the embodiments and drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from context

The cannabinoids are a class of molecules primarily obtained through the extraction of cannabis plant material or via synthetic routes known to those having skill in the art. The various cannabinoids include tetrahydrocannabinol (THC), cannabidiol (CBD), tetrahydrocannabinolic acid (THCA), Cannabidiolic acid (CBDA), cannabinol (CBN), cannabigerol (CBG), cannabichromene (CBC), tetrahydrocannabivarin (THCV), and cannabidivarin (CBDV) as well as others. Various cannabinoids, used alone or in combination have shown a variety of significant biological effects including but not limited to pain relief, anti cancer, anti inflammatory, anti emetic, anti convulsant, and many others.

As used in regards to an SAE-CD-containing composition or formulation according to the invention, the term dilutable refers to a liquid formulation containing SAE-CD and an active agent, such as a cannabinoid, for example, wherein the formulation can be further diluted (with water or dextrose (5%) in water at room temperature, e.g., ambient temperature such as a temperature of about 20°-28° C.) without precipitation, i.e. if precipitation occurs it is less than or equal to about 3% wt. (in other words, precipitation is insignificant), of the active agent while maintaining a clear solution when diluted to a cannabinoid concentration of about 0.5-5 mg/mL. A dilutable SAE-CD and active agent-containing liquid can be diluted with another solution that does not contain SAE-CD and the resulting diluted solution will have a lower concentration of active agent without affecting significant precipitation of the active agent. Accordingly, an SAE-CD and active agent-containing solution that is not dilutable according to the invention will form a significant amount (>3% wt. of active agent) of precipitate when diluted with another solution.

It should be noted that a solution that is not dilutable with water at room temperature may be rendered dilutable with a solution that contains SAE-CD as long as the final molar ratio of CBD to SAE-CD in the diluted solution is within the range as described herein. The invention therefor provides a method of rendering dilutable a previously non-dilutable (as defined herein) CBD-containing solution comprising the step of diluting the previously non-dilutable solution with a second solution containing SAE-CD such that the molar ratio of SAE-CD to the at least one cannabinoid in the diluted solution is ≥10-50

Temperature will likely have an effect upon the dilutability of a solution. In general, the determination of whether or not a solution is dilutable is made at approximately 25° C. or ambient temperature, e.g., 20°-28° C. A solution that is not dilutable at about 25° C. can be made dilutable with water at room temperature by dilution at an elevated temperature, such as >30° C., >40° C., >50° C. or higher. This heated dilution can be performed by diluting the first 25° C. solution with a heated solution or by mixing and heating two solutions which are initially at ambient temperature. Alternatively, the two solutions can be heated separately and then mixed.

Dilutability of an SAE-CD and cannabinoid-containing solution at ambient temperature is particularly important in the clinical setting wherein solutions are not typically heated prior to mixing. Accordingly, the present invention provides solutions of cannabinoids that can be diluted at ambient temperature without the need of a surfactant, organic solvent, soap, detergent or other such compound.

As used herein, a pharmaceutically acceptable liquid carrier is any aqueous medium used in the pharmaceutical sciences for dilution or dissolution of parenteral or enteral formulations.

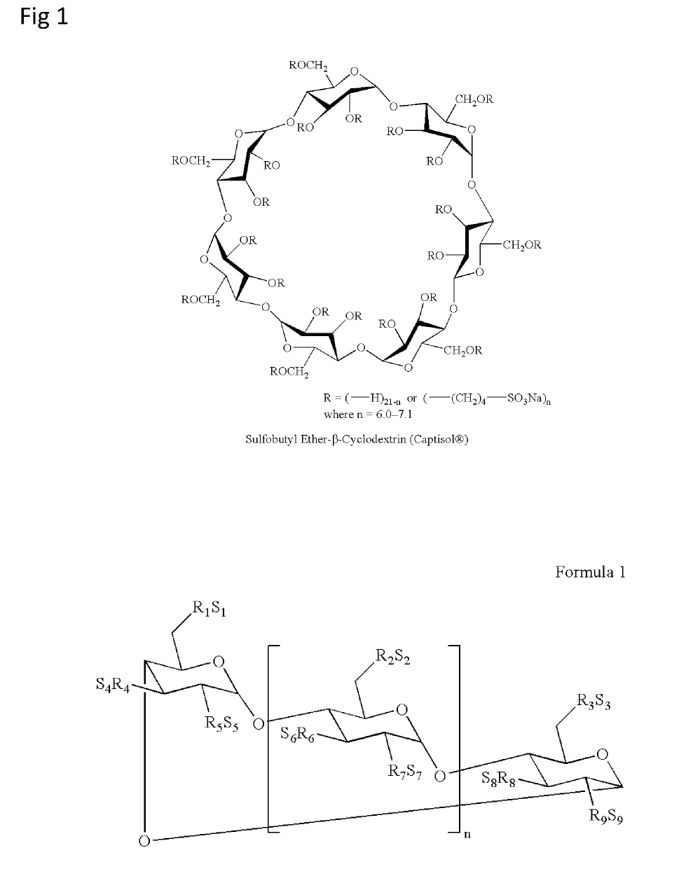

The formulation of the invention comprises at least one cannabionoid and a sulfoalkyl ether cyclodextrin of the formula 1 (see FIG. 1).

The SAE-CD used in the liquid or solid formulation is described in U.S. Pat. Nos. 5,376,645 and 5,134,127 to Stella et al, the entire disclosures of which are hereby incorporated by reference. The preparation process may comprise dissolving the cyclodextrin in aqueous base at an appropriate temperature, e.g., 70° to 80° C., at the highest concentration possible. For example, to prepare the cyclodextrin derivatives herein, an amount of an appropriate alkyl sultone, corresponding to the number of moles of primary CD hydroxyl group present, is added with vigorous stirring to ensure maximal contact of the heterogeneous phase. According to one embodiment, the SAE-CD is SBE-7-β-CD (CAPTISOL® cyclodextrin), or SBE-4-β-CD.

The terms “alkylene” and “alkyl,” as used herein (e.g., in the -0-(C2-C6-alkylene)SO3—group or in the alkylamines), include linear, cyclic, and branched, saturated and unsaturated (i.e., containing one double bond) divalent alkylene groups and monovalent alkyl groups, respectively. The term “alkanol” in this text likewise includes both linear, cyclic and branched, saturated and unsaturated alkyl components of the alkanol groups, in which the hydroxyl groups may be situated at any position on the alkyl moiety. The term “cycloalkanol” includes unsubstituted or substituted (e.g., by methyl or ethyl) cyclic alcohols.

The present invention provides compositions containing a mixture of cyclodextrin derivatives, having the structure set out in formula (I), where the composition overall contains on the average at least 1 and up to 3n+6 alkylsulfonic acid moieties per cyclodextrin molecule. The present invention also provides compositions containing a single type of cyclodextrin derivative, or at least 50% of a single type of cyclodextrin derivative.

The cyclodextrin derivatives of the present invention are obtained as purified compositions, i.e., compositions containing at least 90 wt. % or 95 wt. % of cyclodextrin derivative(s). In a preferred embodiment, purified compositions containing at least 98 wt. % cyclodextrin derivative(s) are obtained.

In some of the compositions of the invention unreacted cyclodextrin has been substantially removed, with the remaining impurities (i.e., <5 wt. % of composition) being inconsequential to the performance of the cyclodextrin derivative-containing composition.

Exemplary SAE-CD derivatives include SBE4-β-CD, SBE7-β-CD, SBE11-β-CD, and SBE4-γ-CD which correspond to SAE-CD derivatives of the formula I wherein n=5, 5, 5 and 6; m is 4; and there are 4, 7, 11 and 4 sulfoalkyl ether substituents present, respectively. It has been found that these SAE-CD derivatives increase the solubility of poorly water soluble drugs, such as CBD, to varying degrees in ways that have not been suggested or disclosed by the prior art.

Exemplary cannabinoids include tetrahydrocannabinol (THC), cannabidiol (CBD), tetrahydrocannabinolic acid (THCA), Cannabidiolic acid (CBDA), cannabinol (CBN), cannabigerol (CBG), cannabichromene (CBC), tetrahydrocannabivarin (THCV), and cannabidivarin (CBDV) as well as others typically described as “minor cannabinoids. In an embodiment a combination of the cannabinoids THC and CBD are used. THC and CBD will be present in varied ratios of between about 1:20 and 20:1. Specific embodiments contemplate ratios of about 1:10 THC to CBD, 1:1 THC to CBD, and 1:10 THC to CBD.

By “therapeutic agent/SAE-CD complex” is generally meant a clathrate or inclusion complex of a sulfoalkyl ether cyclodextrin derivative of the formula (1) and a therapeutic agent. The ratio of therapeutic agent: SAE-CD present in the molecular complex can vary and can be in the range of about 0.33 to about 50, on a molar basis. Thus, the SAE-CD will generally be, but need not be, present in excess of the therapeutic agent. The amount of excess will be determined by the intrinsic solubility of the agent, the expected dose of the agent, and the binding constant for inclusion complexation between the specific drug (agent) and the specific SAE-CD.

By “complexed” is meant “being part of a clathrate or inclusion complex with”, i.e., a complexed therapeutic agent is part of a clathrate or inclusion complex with a sulfoalkyl ether cyclodextrin derivative. By “major portion” is meant at least about 50% by weight of the therapeutic compound. In various specific embodiments, greater than 50%, 60%, 75%, 90% or 95% by weight of the therapeutic agent can be complexed with an SAE-CD while in the pharmaceutical formulation. The actual percent of drug that is complexed will vary according to the complexation equilibrium constant characterizing the complexation of a specific SAE-CD to a specific drug and to the concentrations of SAE-CD and drug available for complexation.

SAE-CD in particular SBE-CD, solubilizes cannabinoids better than other cyclodextrins. Several experiments with other cylclodextrins failed to produce solubility profiles similar to that of the SAE-CD. Accordingly, the present invention provides an improved method of solubilizing cannabinoids comprising the step of including an SAE-CD in a formulation comprising one or more cannabinoids.

As shown in Table 1 the complexation with an SAE-CD, in this case, Captisol has significantly increased the solubility of CBD in water and other aqueous solvents. In some embodiments the addition of water soluble polymers and/or surfactants increases the solubility of the CBD/Captisol mixture. Therefore, in some embodiments the formulations may include the addition of these or other ingredients as solubility enhancers.

Surprisingly we found that Cremophor EL, alone provided even greater solubility enhancement than SAE-CD. Therefore an embodiment of the present invention includes a

TABLE 1

Captisol

| | | 40% | 20% | 0% |
|---|---|---|---|---|
| None | | 2.8 | 0.85 | 0.00026 |
| 0.5% | PVP | 2.948 | 1.175 | |
| | CMC | 3.27 | 0.0145 | |
| | HPMC | 2.68 | 1.32 | |
| | Poloxamer 188 | | 1.411 | 0.133 |
| | Tween 80 | 2.02 | | |
| 1% | PVP | 3.28 | 1.5159 | 0.6025 |
| | CMC | 2.6366 | 1.712 | 0.593 |
| | HPMC | 3.447 | 1.552 | 0.59 |
| | Tween 80 | 2.65 | 2.014 | 4.198 |
| | CrEL | | 4.2767 | 8.96 |
| 2% | PVP | | 0.84 | |
| | CMC | | 0.3652 | |
| | Tween 80 | | 0.72 | |
| 5% | CrEL and EtOH | | 21.5 | 36.22 |
| | CrEL | | 13.51 | 21.92 |
| 10% | CrEL | | 46.705 | 47.85 |
| | EtOH | | 0.92 | N/D |
| | pH 9 | | 0.85 | N/D |
| | pH 4 | | 0.65 | N/D |
| | pH ~2 | | 2.48 | N/D |
| | PBS (pH = 7.4) | 2.3 | 0.72 | N/D |

composition of matter/formulation comprising a cannabinoid, a plurality of cannabinoids, an/or a whole plant cannabis extract, cremophor EL (0.1%-20%) and an aqueous solvent.

It should be understood that other SAE-CD compounds of the formula 1 may be used in the liquid formulation of the invention. These other SAE-CD formulations differ from SBE7-β-CD in their degree of substitution by sulfoalkyl groups, the number of carbons in the sulfoalkyl chain, their molecular weight, the number of glucopyranose units contained in the base cyclodextrin used to form the SAE-CD and or their substitution patterns. In addition, the derivatization of β-cyclodextrin with sulfoalkyl groups occurs in a controlled, although not exact manner. For this reason, the degree of substitution is actually a number representing the average number of sulfoalkyl groups per cyclodextrin (for example, SBE7-β-CD, has an average of 7 substitutions per cyclodextrin). In addition, the regiochemistry of substitution of the hydroxyl groups of the cyclodextrin is variable with regard to the substitution of specific hydroxyl groups of the hexose ring. For this reason, sulfoalkyl substitution of the different hydroxyl groups is likely to occur during manufacture of the SAE-CD, and a particular SAE-CD will possess a preferential, although not exclusive or specific, substitution pattern. Given the above, the molecular weight of a particular SAE-CD may vary from batch to batch and will vary from SAE-CD to SAE-CD. All of these variations can lead to changes in the complexation equilibrium constant K1:1 which in turn will affect the required molar ratios of the SAE-CD to the at least one cannabinoid. The equilibrium constant is also somewhat variable with temperature and allowances in the ratio are required such that the agent remains solubilized during the temperature fluctuations that can occur during manufacture, storage, transport, and use. The equilibrium constant is also variable with pH and allowances in the ratio are required such that the agent remains solubilized during pH fluctuations that can occur during manufacture, storage, transport, and use. The equilibrium constant is also variable by the presence of other excipients (e.g., buffers, preservatives, antioxidants) Accordingly, the ratio of SAE-CD/cannabinoid may need to be varied (±from the ratios set forth herein) in order to compensate for the above-mentioned variables.

The invention also provides a pharmaceutical kit comprising a first container containing a liquid vehicle and a second container containing a reconstitutable solid pharmaceutical composition as described above. The liquid vehicle comprises an aqueous liquid carrier such as water, dextrose, saline, lactated Ringer's solution, or any other pharmaceutically acceptable aqueous liquid vehicles for the preparation of a liquid pharmaceutical compound.

A complexation-enhancing agent can be added to the aqueous liquid formulation of the invention. A complexation-enhancing agent is a compound, or compounds, that enhance(s) the complexation of cannabinoids with the SAE-CD. When the complexation-enhancing agent is present, the required ratio of SAE-CD to the cannabinoid may need to be changed such that less SAE-CD is required. Suitable complexation enhancing agents include one or more pharmacologically inert water soluble polymers, hydroxy acids, and other organic compounds typically used in liquid formulations to enhance the complexation of a particular agent with cyclodextrins. Suitable water soluble polymers include water soluble natural polymers, water soluble semisynthetic polymers (such as the water soluble derivatives of cellulose) and water soluble synthetic polymers. The natural polymers include polysaccharides such as inulin, pectins, algin derivatives and agar, and polypeptides such as casein and gelatin. The semi-synthetic polymers include cellulose derivatives such as methylcellulose, hydroxyethyl cellulose, hydroxypropyl cellulose, their mixed ethers such as hydroxypropyl methylcellulose and other mixed ethers such as hydroxyethyl ethylcellulose, hydroxypropyl ethylcellulose, hydroxypropyl methylcellulose phthalate and carboxymethylcellulose and its salts, especially sodium carboxymethylcellulose. The synthetic polymers include polyoxyethylene derivatives (polyethylene glycols) and polyvinyl derivatives (polyvinyl alcohol, polyvinylpyrrolidone and polystyrene sulfonate) and various copolymers of acrylic acid (e.g. carbomer). Suitable hydroxy acids include by way of example, and without limitation, citric acid, malic acid, lactic acid, and tartaric acid and others known to those of ordinary skill in the art.

A solubility-enhancing agent can be added to the aqueous liquid formulation of the invention. A solubility-enhancing agent is a compound, or compounds, that enhance(s) the solubility of the at least one cannabinoid(s) in the liquid formulation. When a complexation-enhancing agent is present, the ratio of SAE-CD to the cannabinoid(s) may need to be changed such that less SAE-CD is required. Suitable solubility enhancing agents include one or more organic solvents, detergents, soaps, surfactants and other organic compounds typically used in parenteral formulations to enhance the solubility of a particular agent. Suitable organic solvents include, for example, ethanol, glycerin, polyethylene glycols, propylene glycol, poloxomers, and others known to those of ordinary skill in the art.

It should be understood, that compounds used in the pharmaceutical arts generally serve a variety of functions or purposes. Thus, if a compound named herein is mentioned only once or is used to define more than one term herein, its purpose or function should not be construed as being limited solely to that named purpose(s) or function(s). Although not necessary, the formulation of the present invention may include a preservative, antioxidant, buffering agent, acidifying agent, alkalizing agent, antibacterial agent, antifungal agent, solubility-enhancing agent, complexation enhancing agent, solvent, electrolyte, salt, water, glucose, stabilizer, tonicity modifier, antifoaming agent, oil, bulking agent, cryoprotectant, or a combination thereof.

As used herein, the term “alkalizing agent” is intended to mean a compound used to provide alkaline medium for product stability. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, diethanolamine, organic amine base, alkaline amino acids and trolamine and others known to those of ordinary skill in the art.

As used herein, the term “acidifying agent” is intended to mean a compound used to provide an acidic medium for product stability. Such compounds include, by way of example and without limitation, acetic acid, acidic amino acids, citric acid, fumaric acid and other alpha hydroxy acids, hydrochloric acid, ascorbic acid, phosphoric acid, sulfuric acid, tartaric acid and nitric acid and others known to those of ordinary skill in the art.

As used herein, the term “preservative” is intended to mean a compound used to prevent the growth of microorganisms. Such compounds include, by way of example and without limitation, benzalkonium chloride, benzethonium chloride, benzoic acid, benzyl alcohol, cetylpyridinium chloride, chlorobutanol, phenol, phenylethyl alcohol, phenylmercuric nitrate, phenylmercuric acetate, thimerosal, metacresol, myristylgamma picolinium chloride, potassium benzoate, potassium sorbate, sodium benzoate, sodium propionate, sorbic acid, thymol, and methyl, ethyl, propyl, or butyl parabens, citric acid, EDTA, ascorbic acid −6—palmitate and others known to those of ordinary skill in the art.

As used herein, the term “antioxidant” is intended to mean an agent which inhibits oxidation and thus is used to prevent the deterioration of preparations by the oxidative process. Such compounds include by way of example and without limitation, acetone, sodium bisulfate, ascorbic acid, ascorbyl palmitate, citric acid, butylated hydroxyanisole, butylated hydroxytoluene, hydrophosphorous acid, monothioglycerol, propyl gallate, sodium ascorbate, sodium citrate, sodium sulfide, sodium sulfite, sodium bisulfate, sodium formaldehyde sulfoxylate, thioglycolic acid, sodium metabisulfite, EDTA (edetate), pentetate and others known to those of ordinary skill in the art.

As used herein, the term “buffering agent” is intended to mean a compound used to resist change in pH upon dilution or addition of acid or alkali. Such compounds include, by way of example and without limitation, acetic acid, sodium acetate, adipic acid, benzoic acid, sodium benzoate, citric acid, maleic acid, monobasic sodium phosphate, dibasic sodium phosphate, lactic acid, tartaric acid, glycine, potassium metaphosphate, potassium phosphate, monobasic sodium acetate, sodium bicarbonate, sodium tartrate and sodium citrate anhydrous and dihydrate and others known to those of ordinary skill in the art.

As used herein, the term “stabilizer” is intended to mean a compound used to stabilize a therapeutic agent against physical, chemical, or biochemical process that would otherwise reduce the therapeutic activity of the agent. Suitable stabilizers include, by way of example and without limitation, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art.

As used herein, the term “tonicity modifier” is intended to mean a compound or compounds that can be used to adjust the tonicity of the liquid formulation. Suitable tonicity modifiers include glycerin, lactose, mannitol, dextrose, sodium chloride, sodium sulfate, sorbitol, trehalose and others known to those or ordinary skill in the art. In one embodiment, the tonicity of the liquid formulation approximates that of the tonicity of blood or plasma.

As used herein, the term “antifoaming agent” is intended to mean a compound or compounds that prevents or reduces the amount of foaming that forms on the surface of the liquid formulation. Suitable antifoaming agents include by way of example and without limitation, dimethicone, simethicone, octoxynol and others known to those of ordinary skill in the art.

As used herein, the term “bulking agent” is intended to mean a compound used to add bulk to the reconstitutable solid and/or assist in the control of the properties of the formulation during preparation. Such compounds include, by way of example and without limitation, dextran, trehalose, sucrose, polyvinylpyrrolidone, lactose, inositol, sorbitol, dimethylsulfoxide, glycerol, albumin, calcium lactobionate, and others known to those of ordinary skill in the art.

As used herein, the term “cryoprotectant” is intended to mean a compound used to protect an active therapeutic agent from physical or chemical degradation during lyophilization. Such compounds include, by way of example and without limitation, dimethyl sulfoxide, glycerol, trehalose, propylene glycol, polyethylene glycol, and others known to those of ordinary skill in the art.

As used herein, the term “solubilizing agent” is intended to mean a compound used to assist and or increase the solubility of a compound going into solution. Such compounds include, by way of example and without limitation, glycerin, glycerol, polyethylene glycol, propylene glycol and others known to those of ordinary skill in the art.

The formulation of the invention can also include water, glucose or saline and combinations thereof. In particular embodiments, the formulation includes water, saline, and glucose.

The pH of the liquid formulation will generally range from about pH 3.0 to about pH 9.0; however, liquid formulations having higher or lower pH values can also be prepared. It is contemplated that cannabinoid stability can be increased by optimizing the pH as well as the SAE-CD to cannabinoid concentration.

The liquid formulation of the invention can be provided in an ampoule, syringe, bottle, bag, vial or other such container typically used for parenteral formulations.

The liquid formulation of the invention can be prepared by numerous different methods. According to one method, a first aqueous solution comprising SAE-CD is prepared. Then, a second solution comprising a cannabinoid is prepared. Finally, the first and second solutions are mixed to form the liquid formulation. The first and second solutions can independently comprise other excipients and agents described herein. Additionally, the second solution can be water and/or an organic solvent-base solution. Alternatively the second solution can be a substantially pure cannabinoid oil, comprising 1 or more purified cannabinoids. Another method of preparation is similar to the above-described method except that the cannabinoid(s) are added directly to the first solution without the formation of a second solution. A third method of preparing the liquid formulation is similar to the above-described first method except that the SAE-CD is added directly to a second solution containing the cannabinoid(s) without formation of the first solution. A fourth method of preparing the liquid formulation comprises the steps of adding an ethanolic solution comprising one or more cannabinoids to a powdered or particulate SAE-CD and mixing the solution until the SAE-CD has dissolved, then allowing the ethanol to evaporate and finally reconstituting the SAE-CD—cannabinoid in an aqueous solution. A fifth method of preparing the liquid formation comprises the steps of adding the cannabinoid(s) directly to the powdered or particulate SAE-CD and then adding an aqueous solution and mixing until the SAE-CD and cannabinoids have dissolved. A sixth method for preparing the liquid formation comprises the steps of heating either the first solution or heating the second solution, or heating a combination thereof of any solutions described in the above methods followed by the step of cooling the respectively heated solution. A seventh method for preparing the liquid formation comprises the step of adjusting the pH of either the first solution or adjusting the pH of the second solution or adjusting the pH of a combination of either solutions described in any of the above methods. An eighth method comprises the steps of creating the liquid formulation by any of the above-described methods followed by the step of isolating a solid material by lyophilization, spray-drying, spray freeze-drying, vacuum-drying, antisolvent precipitation or a process utilizing a supercritical or near supercritical fluid. Any of the above solutions can contain other pharmaceutical excipients or ingredients as described herein. In some embodiments sonication steps are added to the above described methods to increase complexation.

Specific embodiments of the method of preparing the liquid formulation include those wherein the method further comprises the step of: 1) sterile filtering the formulation through a filtration medium wherein the pore size is about 0.22 μm or smaller; 2) sterilizing the liquid formulation by irradiation; 3) sterilizing the liquid formulation by treatment with ethylene oxide; 4) isolating a sterile powder from the sterilized liquid formulation; 5) purging the liquid with an inert gas to reduce the amount of dissolved oxygen in the liquid; and/or 6) one or more of the solutions used to prepare the liquid formulation is heated.

The first and second formulations can be mixed and formulated as a liquid dosage form prior to administration to a subject. Either one or both of the first and second pharmaceutical compositions can comprise additional pharmaceutical components.

The liquid formulation of the invention can be provided in a kit. The kit will comprise a first pharmaceutical composition comprising an SAE-CD and a second pharmaceutical composition comprising one or more cannabinoids. The first and second formulations can be mixed and formulated as a liquid dosage form prior to administration to a subject. Either one or both of the first and second pharmaceutical compositions can comprise additional pharmaceutical excipients. The kit is available in various forms.

In a first kit, the first and second pharmaceutical compositions are provided in separate containers or separate chambers of a container having two or more chambers. The first and second pharmaceutical compositions may be independently provided in either solid or powder or liquid form. For example, the SAE-CD can be provided in a reconstitutable powder form and the cannabinoid(s) be provided in an oil form, or in a powder form where the chemical properties of the selected cannabinoid(s) allow. According to one embodiment, the kit would further comprise a pharmaceutically acceptable liquid carrier used to suspend and dissolve the first and/or second pharmaceutical compositions. Alternatively, a liquid carrier is independently included with the first and/or second pharmaceutical composition. The liquid carrier, however, can also be provided in a container or chamber separate from the first and second pharmaceutical compositions. As above, the first pharmaceutical composition, the second pharmaceutical composition and the liquid carrier can independently comprise a preservative, an antioxidant, a buffering agent, an acidifying agent, saline, glucose, an electrolyte, another therapeutic agent, an alkalizing agent, an antimicrobial agent, an antifungal agent, solubility enhancing agent or a combination thereof. The liquid formulation of the invention can be provided as a dosage form including a pre-filled vial, pre-filled bottle, pre-filled syringe, pre-filled ampoule, or plural ones thereof. Generally, a pre-filled container will contain at least a unit dosage form of the cannabinoid agent.

Specific embodiments of the kit include those wherein: 1) the first and second pharmaceutical compositions are contained in separate containers or separate chambers of a container having two or more chambers; 2) the kit further comprises a separate pharmaceutically acceptable liquid carrier; 3) a liquid carrier is included with the first and/or second pharmaceutical composition; 4) containers for the pharmaceutical compositions are independently selected at each occurrence from an evacuated container, a syringe, bag, pouch, ampule, vial, bottle, or any pharmaceutically acceptable device known to those skilled in the art for the delivery of liquid formulations; 5) the first pharmaceutical composition and/or second pharmaceutical composition and/or liquid carrier further comprises an antioxidant, a buffering agent, an acidifying agent, a solubilizing agent, a complexation enhancing agent, saline, dextrose, lyophilizing aids (for example, bulking agents or stabilizing agents), an electrolyte, another therapeutic agent, an alkalizing agent, an antimicrobial agent, an antifungal agent or a combination thereof; 6) the kit is provided chilled; 8) the liquid carrier and/or chamber has been purged with a pharmaceutically acceptable inert gas to remove substantially all of the oxygen dissolved in the liquid carrier; 9) the chambers are substantially free from oxygen; 10) the liquid carrier further comprises a buffering agent capable of maintaining a physiologically acceptable pH; 11) the chambers and solutions are sterile; 12) a diluent included in the kit comprises SAE-CD and is used to dilute a formulation that is non-dilutable in D5W at room temperature.

The term “unit dosage form” is used herein to mean a single or multiple dose form containing a quantity of the active ingredient and the diluent or carrier, said quantity being such that one or more predetermined units are normally required for a single therapeutic administration. In the case of multiple dose forms, such as liquid-filled ampoules, said predetermined unit will be one fraction such as a half or quarter of the multiple dose form. It will be understood that the specific dose level for any patient will depend upon a variety of factors including the indication being treated, therapeutic agent employed, the activity of therapeutic agent, severity of the indication, patient health, age, sex, weight, diet, and pharmacological response, the specific dosage form employed and other such factors.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “patient” is taken to mean warm blooded animals such as mammals, for example, cats, dogs, mice, guinea pigs, horses, bovine cows, sheep, and humans.

The liquid formulation of the invention will comprise an effective amount of one or more cannabinoids. By the term “effective amount”, it is understood that a therapeutically effective amount is contemplated. A therapeutically effective amount is the amount or quantity of one or more cannabinoids that is sufficient to elicit the required or desired therapeutic response, or in other words, the amount that is sufficient to elicit an appreciable biological response when administered to a subject.

The present compositions/formulations can be used to address a variety of diseases and/or medical conditions. In some embodiments, the diseases include, but are not limited to, Acquired Hypothyroidism, Acute Gastritis, Acute Pain, Agoraphobia, AIDS Related Illness, Alcohol Abuse, Alcoholism, Alopecia Areata, Alzheimer's Disease, Amphetamine Dependency, Amyloidosis, Amyotrophic Lateral Sclerosis (ALS), Angina Pectoris, Ankylosis, Anorexia, Anorexia Nervosa, Anxiety Disorders, any chronic medical symptom that limits major life activities, any Chronic Medical Symptom that Limits Major Life Activities, Arteriosclerotic Heart Disease, Arthritis, Arthritis (Rheumatoid), Arthropathy, gout, Asthma, Attention Deficit Hyperactivity Disorder (ADD/ADHD), Autism/Asperger's, Autoimmune Disease, Back Pain, Back Sprain, Bell's Palsy, Bipolar Disorder, Brain Tumor, Breakthrough Pain, Malignant, Bruxism, Bulimia, Cachexia, Cancer, Carpal Tunnel Syndrome, Cerebral Palsy, Cervical Disk Disease, Cervicobrachial Syndrome, Chemotherapy Chronic Fatigue Syndrome, Chronic Pain, Chronic renal failure, Cocaine Dependence, Colitis, Conjunctivitis, Constipation, Crohn's Disease, Cystic Fibrosis, Damage to Spinal Cord Nervous Tissue, Darier's Disease, Degenerative Arthritis, Degenerative Arthropathy, Delirium Tremens, Dermatomyositis, Diabetes, Diabetic Neuropathy, Diabetic Peripheral Vascular Disease, Diarrhea, Diverticulitis, Dysthymic Disorder, Eczema, Emphysema, Emphysema, Endometriosis, Epidermolysis Bullosa, Epididymitis, Epilepsy, Felty's Syndrome, Fibromyalgia, Friedreich's Ataxia, Gastritis, Genital Herpes, Glaucoma, Glioblastoma Multiforme, Graves Disease, Cluster Headaches, Migraine Headaches, Tension Headaches, Hemophilia A, Henoch-Schonlein Purpura, Hepatitis C, Hereditary Spinal Ataxia, HIV/AIDS, Hospice Patients, Huntington's Disease, Hypertension, Hypertension, Hyperventilation, Hypoglycemia, Impotence, Inflammatory autoimmune-mediated arthritis, Inflammatory Bowel Disease (IBD), Insomnia, Intermittent Explosive Disorder (IED), Intractable Pain, Intractable Vomiting, Lipomatosis, Lou Gehrig's Disease, Lyme Disease, Lymphoma, Major Depression, Malignant Melanoma, Mania, Melorheostosis, Meniere's Disease, Motion Sickness, Mucopolysaccharidosis (MPS), Multiple Sclerosis (MS), Muscle Spasms, Muscular Dystrophy, Myeloid Leukemia, Nail-Patella Syndrome, Nightmares, Obesity, Obsessive Compulsive Disorder, Opiate Dependence, Osteoarthritis, Panic Disorder, Parkinson's Disease, Peripheral Neuropathy, Peritoneal Pain, Persistent Insomnia, Porphyria, Post Polio Syndrome (PPS), Post-traumatic arthritis, Post-Traumatic Stress Disorder (PTSD), Premenstrual Syndrome (PMS), Prostatitis, Psoriasis, Pulmonary Fibrosis, Quadriplegia, Radiation Therapy, Raynaud's Disease, Reiter's Syndrome, Restless Legs Syndrome (RLS), Rheumatoid Arthritis, Rheumatoid Arthritis, Rheumatoid Arthritis, Rosacea, Schizoaffective Disorder, Schizophrenia, Scoliosis, Sedative Dependence, Seizures, Senile Dementia, Severe Nausea, Shingles (Herpes Zoster), Sinusitis, Skeletal Muscular Spasticity, Sleep Apnea, Sleep Disorders, Spasticity, Spinal Stenosis, Sturge-Weber Syndrome (SWS), Stuttering, Tardive Dyskinesia (TD), Temporomandibular joint disorder (TMJ), Tenosynovitis, Terminal Illness, Thyroiditis, Tic Douloureux, Tietze's Syndrome, Tinnitus, Tobacco Dependence, Tourette's Syndrome, Trichotillomania, Viral Hepatitis, Wasting Syndrome, Whiplash, Wittmaack-Ekbom's Syndrome, Writers' Cramp, nausea, vomiting, premenstrual syndrome, unintentional weight loss, insomnia, and lack of appetite, spasticity, painful conditions, especially neurogenic pain, movement disorders, asthma, glaucoma, adrenal disease, inflammatory bowel disease, migraines, fibromyalgia, and related conditions, multiple sclerosis, spinal cord injuries. It exhibits antispasmodic and muscle-relaxant properties as well as stimulates appetite. Other studies state that cannabis or cannabinoids may be useful in treating alcohol abuse, amyotrophic lateral sclerosis, collagen-induced arthritis, asthma, atherosclerosis, bipolar disorder, colorectal cancer, HIV-Associated Sensory Neuropathy, depression, dystonia, epilepsy, digestive diseases, gliomas, hepatitis C, Huntington's disease, leukemia, skin tumors, methicillin-resistant Staphylococcus aureus (MRSA), Parkinson's disease, pruritus, posttraumatic stress disorder (PTSD), psoriasis, sickle-cell disease, sleep apnea, and anorexia nervosa.

While the present disclosure includes many embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

With respect to the above, it is to be understood that the invention is not limited in its application to the details of construction and to the arrangement of the components listed or the steps set forth in the description or illustrated in the drawings. The various apparatus and methods of the disclosed invention are capable of other embodiments, and of being practiced and carried out in various ways that would be readily known to those skilled in the art, given the present disclosure. Further, the terms and phrases used herein are for descriptive purposes and should not be construed as in any way limiting.

As such, those skilled in the art will appreciate that the conception upon which this disclosure is based may be utilized as a basis for designing other inventions with similar properties. It is important therefore that the embodiments, objects, and claims herein, be regarded as including such equivalent construction and methodology insofar as they do not depart from the spirit and scope of the present invention.

What is claimed is:

1. A clear ready-to-inject liquid formulation comprising BE7-β-CD and at least one cannabinoid, and the at least one cannabinoid is present in amount of 0.5-10 mg/mL.

2. A water soluble clear liquid formulation comprising one or more cannabinoids and a sulfoalkyl ether cyclodextrin (SAE-CD) wherein the liquid formulation is dilutable with water at ambient temperature without significant precipitation of the one or more cannabinoids, and wherein the SAE-CD is a compound or mixture of compounds of the formula 1.

3. A clear water dilutable liquid formulation comprising SBE7-β-CD and at least one cannabinoid, wherein the cannabinoid is present in an amount of greater than about 1.5 mg/ml.

4. A method for creating a water soluble cannabinoid formulation comprising the steps of mixing a an acqueous solution containing an SAE-CD with at least one purified cannabinoid.

5. A method for treating a subject with a cannabinoid comprising the steps of injecting the subject with a clear ready-to-inject liquid formulation comprising BE7-β-CD and at least one cannabinoid, and the at least one cannabinoid is present in amount of 0.5-10 mg/mL.

6. The formulation of claim 1 wherein the at least one cannabinoid is cannabidiol (CBD).

7. The formulation of claim 1 wherein the at least one cannabinoid is tetrahydracannabinol(THC).

8. The formulation of claim 1 wherein the at least one cannabinoid is a combination of THC and CBD.

9. The formulation of claim 8 wherein the ratio of THC to CBD is between about 1:10 and 10:1.
